(19) United States
(12) Patent Application Publication
(10) Pub. No.: US 2021/0334922 A1
(43) Pub. Date: Oct. 28, 2021

(54) INTEGRATED COMBAT VETERAN RECOVERY AND RESILIENCY PROGRAM

(71) Applicant: Magnus Johnson, West Linn, OR (US)

(72) Inventor: Magnus Johnson, West Linn, OR (US)

(21) Appl. No.: 16/859,748

(22) Filed: Apr. 27, 2020

(51) Int. Cl. G06Q 50/22; G16H 50/30; A61B 5/0205; A61B 5/00; A61B 5/16

(52) U.S. Cl. CPC G06Q 50/22; G16H 50/30; A61M 21/02; A61B 5/4064; A61B 5/165; A61B 5/0205

(57) ABSTRACT

A year-long, four-phase integrated, holistic recovery and resiliency program that heals the emotional scars of the soul of a combat veteran or other individual resulting from PTSD or combat related TBI, each of the four phases themselves comprising four healing aspects, namely emotional, physical, spiritual and nutritional healing, and each of these phases occurring in a logical progression and integrating with the appropriate aspects of recovery for each such phase that results in an end healing effect on the combat veteran greater than the sum of the individual aspects.

### BACKGROUND

The present disclosure relates generally to a COMBAT VETERAN PROGRAM. In particular, an INTEGRATED COMBAT VETERAN RECOVERY AND RESILIENCY PROGRAM (“the program”) is described.

Known post-traumatic stress disorder (PTSD) and traumatic brain injury (TBI) emotional healing and management programs are not entirely satisfactory for the range of combat veteran treatment applications in which they are employed. For example, existing programs may address one or another issue at a time. However, there is currently nothing that presents a holistic program that addresses physical, nutritional, spiritual and emotional needs simultaneously, integrated into a single platform, in a way that synergistically improves upon emotionally healing and managing the combat veteran's PTSD or TBI. The most recent numbers available from Department of Veterans Affairs (from 2019) states the following: In 2017, the suicide rate for Combat Veterans was 1.5 times the rate for non-Veteran adults, after adjusting for population differences in age and sex. Indeed, conventional programs must not have worked effectively since the number of combat veteran suicides remains higher than the rate for the general population. The instant program has never had a suicide amongst its veterans.

Thus, there exists a need for programs that improve upon and advance the design of known programs. Examples of new and useful programs relevant to the needs existing in the field are discussed below.

### SUMMARY

The present disclosure is directed to a system and method to facilitate an integrated combat veteran recovery and resiliency program for reintegrating veterans into civilian life. A primary objective of the combat veteran recovery and resiliency program is to show each veteran that they have agency over their level of stress. Another objective is to demonstrate that the veteran can take control of what are thought to be involuntary symptoms.

The year-long program includes:

A. an emotional health program through access to a licensed counselor and certified health coach, reading material, biofeedback devices and self-examination through journaling by the combat veteran, formation of a cohort of combat veterans who will proceed through the recovery and resiliency program together, communication to and feedback from the cohort concerning actions and feelings of the combat veteran;

B. a physical health program comprising paid membership through group fitness-based gyms, a holistic health coach, biofeedback devices including a fitness tracking device;

C. a spiritual health program facilitated in part through interacting with other members of the cohort, using biofeedback devices that ease the combat veteran into an uplifting meditation practice; and

D. a nutritional health program through an online library of nutritional resources along with regular deliveries of targeted, science-backed food supplements.

These four aspects in the combat veteran recovery and resiliency program, used simultaneously, are presented over a year's time in a planned, graduated, synergistic and well curated manner to provide a robust emotional health program. Alone, each of the components may have some valuable effects when used as directed. But when used together, the effects are magnified more than the sum of their parts.

### BRIEF DESCRIPTION OF THE DRAWINGS

FIG. 1 is a block diagram conceptually illustrating four exemplary phases of a combat veteran recovery and resiliency program.

FIG. 2 is a block diagram of elements of Phase 1 of the combat veteran recovery and resiliency program 100.

FIG. 3 is a block diagram of elements of Phase 2 of the combat veteran recovery and resiliency program.

FIG. 4 is a block diagram of elements of Phase 3 of the combat veteran recovery and resiliency program.

FIG. 5 is a block diagram of elements of Phase 5 of the combat veteran recovery and resiliency program.

FIG. 6 is a block diagram of the analysis system of FIG. 1 in an operating environment.

### DETAILED DESCRIPTION

The disclosed programs will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various programs are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“PTSD” means post-traumatic stress disorder.

“TBI” means traumatic brain injury.

“Combat Veteran” means an individual who has served in the armed forces and has seen active combat.

“Cohort” means a group of recovering combat veterans working together through the phases of the program.

Integrated Combat Veteran Recovery And Resiliency Program

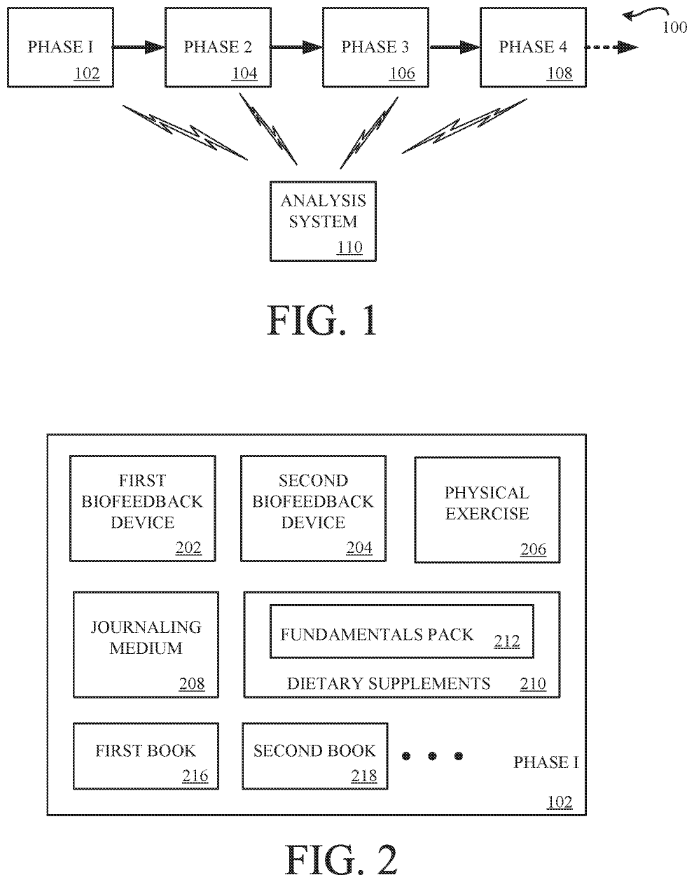

With reference to the figures, embodiments of the combat veteran recovery and resiliency program 100 will now be described. FIG. 1 is a block diagram conceptually illustrating four exemplary phases 102, 104, 106 and 108 of a combat veteran recovery and resiliency program 100. The combat veteran recovery and resiliency programs 100 discussed herein function to provide an emotional healing program that teaches the combat veteran that there are mechanisms and habits that can ameliorate the symptoms of PTSD and TBI. In this example preferred embodiment, the combat veteran recovery and resiliency program 100 is defined by four sequential phases 102, 104, 106 and 108. Alternative embodiments of the combat veteran recovery and resiliency program 100 may have more than, or less than, the example four phases. The combat veteran recovery and resiliency program 100 further comprises an analysis system 110 that is configured to acquire information from various biometric devices employed by the four sequential phases 102, 104, 106 and 108. Preferably, the biometric devices are in communication with the analysis system 110 using a suitable wireless communication signal technology or a suitable wire-based connector.

The reader will appreciate from the figures and description below that the presently disclosed programs address many of the shortcomings of conventional recovery and resiliency programs.

Contextual Details

The features of items used in conjunction with the programs described herein will first be described to provide context and to aid the discussion of the various embodiments of the combat veteran recovery and resiliency programs 100. The objective is that every element of the combat veteran recovery and resiliency program 100 contributes to a veteran's better sleep, mood, sense of well being and decreased stress.

Phase 1

FIG. 2 is a block diagram of elements of Phase 1 (reference numeral 102) of the combat veteran recovery and resiliency program 100. Phase 1 employs a first biofeedback device 202 to stimulate and cause parasympathetic nervous system activation of the user who is an individual participating in the combat veteran recovery and resiliency program 100 to induce a rich and meditative alpha and theta brain wave state. A non-limiting example of a first biofeedback device 202 is the NuCalm® device.

Combat Veterans and individuals who live with chronic stress have an impaired ability to transition their physiology into a parasympathetic dominant state: The fight or flight system, which runs counter to the parasympathetic system, is turned on far too often. Sleep and daily functions are diminished when there is no healthy outlet to relieve hypervigilance. This first example biofeedback device 202 is sold under the brand name NuCalm® and is a patented system (U.S. Pat. No. 9,079,030 issued on Jul. 14, 2015) that induces relaxation in the same way that meditation can provide relaxation, namely by causing increased alpha and theta brain wave activity. Alternative embodiments may employ other types of first biofeedback devices 202 that have an activation effect on the individual's parasympathetic nervous system.

There are many modalities to increase parasympathetic nervous system (PNS) tone. The difference with the first biofeedback devices, such as the NuCalm® device, is that not only does it help to put the body in a repair mode, it takes another step forward by placing the mind in the alpha and theta brainwave activity. To reach this mental state, one would either need to be in a lucid dreaming phase or be a very well practiced meditator. While meditation is certainly a component of this program, this device effectively places the user in that state via a “non-linear oscillating algorithm.” During theta brain wave activity, repair processes are unlocked in the body that aren't active in any other state. Theta state promotes cell toxin clean up and mitochondrial genesis. When a Warrior cannot sleep well, they're missing out on these critical healing components.

A second biofeedback device 204 is used to track recovery from stress and to perform sleep analysis, and send the data generated to the health coach for review and analysis to the analysis system 110.

While no one metric can demonstrate objective long-term healing, the objective of the second biofeedback device 204, such as, but not limited to, a Garmin® fēnix® device, is to open a window into the physiological response of the individual to stress. This non-limiting second biofeedback device 204 is sold under the Garmin® fēnix® brand and it is worn on the wrist of the veteran like a watch.

A unique feature of the fēnix® line is its use of optical heart rate monitoring. By measuring the beat to beat gaps in heart rate, the device can harness the power of heart rate variability (HRV) to understand the state of an individual's real-time nervous system function. The nervous system reflects the body's stress as a screen projects a movie. While there is more to the stress equation than nervous system function, tapping into HRV is a cutting edge method to measure a bodily function that is directly influenced by stress levels. The Garmin® fēnix® has some of the most advanced HRV/optical HR technology available. The user doesn't have to wear a strap to get data so it makes it a lot easier to collect data and use the device than devices that require straps. Garmin®'s software combined with the fēnix® helps to gauge how the rest of the program is affecting stress and sleep overtime, two incredibly important measures of success.

Based on the data generated by this second biofeedback device 204, the veteran can begin to understand, objectively, how the veteran's actions and behaviors can affect the veteran's own biometrics in real time. The power to understand that the veteran has agency over a stress response is hard to value, but a primary objective of the program is to show each veteran that they have agency over their level of stress.

PTSD sufferers often feel as though they have no control of their systems. Their hearts race in flashback episodes, they have difficulty sleeping, and often many internal bodily functions compromised. The program is designed to demonstrate that the veteran can take control of what are through to be involuntary symptoms.

This analysis system 110 also has a platform that allows aggregation of data to potentially spot trends in metrics like sleep and stress scores and this indicates the efficacy of the program.

Engaging in physical exercise program with members of the cohort at a gym or fitness center.

Physical exercise, conceptually illustrated as block 206 in FIG. 2, has long been known to lift one's mood or spirit, decrease tension and stress, and working out with a cohort of people going through the same challenges strengthens that mood lifting further and at a deeper level. It is a vital part of the program generally. Specifically, group exercise done with the cohort provides mutual support to all concerned. Trust and focus in purpose strengthen within the cohort when the members work together toward a common goal. Exercise itself, spirit and mood improvement, cohort interdependence and support therefore all result from group exercise, and the gym setting provides a routine, predictable in practice, from which all who engage will enjoy benefit. Hormonal rebalancing and social connectedness are cornerstones of this program's success. Few outlets are as powerful at reinvigorating these components as a community gym membership.

Recording spiritual and emotional growth in a journal designed to capture these experiences.

Taking control of fight or flight response requires intense habit reformation. Gratitude journaling and written emotional processing by themselves offer a powerful, scientifically proven technique to help an individual become more aware of their emotions and potentially jumpstart emotional healing.

Phase 1 employs a journaling medium 208. The Monk Manual is the preferred journaling medium 208 offered in embodiments of the combat veteran recovery and resiliency program, though any suitable journaling medium system or method may be used in alternative embodiments. Each page of the manual 208 provides space and cues for: recording the priorities of the day; a list of “to-dos” for routines and little things; a daily gratitude list to be completed by the participant to focus on uplifting thoughts and feelings and has been shown to aid in sleep; an “I'm looking forward to” section provides space for particular item and sends powerful signals to the veteran's unconsciousness saying that life is worth living.

This is a missing piece in many people's lives struggling with TBI because the brain effectively loses the will to live for lack of healthy stimulation and objectives to look forward to; a daily schedule helps take control over the daytime hours and provides structure so each hour can be lived with intention; a “habit” section reinforces a particular habit that the user works towards establishing on a monthly basis; a “theme’ section allows the user to choose a theme to focus on and live by; there is space for listing ways the user may give to others that helps take the user out of self-centered thinking; a “highlights” section provides space for listing the high points of the day; three sections focus the user on what they did best that day, what made them feel uneasy and what they can do to improve for the next day. And of course, there is a section at the bottom of the page for miscellaneous notes, doodles or thoughts, recording food consumption, whatever the veteran needs.

Each journaling medium 208, such as the non-limiting example Monk Manual, preferably covers 90 days. When the four sequential phases 102, 104, 106 and 108 are spaced apart by three months, a 90 day journaling medium 208 fits in and coordinates perfectly with the veterans' quarterly recovery and resiliency program 100. The journaling medium 208 is provided in Phases 1 through 4, inclusive. The journaling medium 208 also provides a record that the veteran can use for health coach and counselor sessions to help the veteran see patterns of thinking and living from day to day. Writing in these categories helps the veteran record their actions, and then reflection on them lets participants see how their actions that day help them feel more at rest. Testimonials for the journal from users as well as the research team attest to the Monk Manual's effectiveness.

Dietary Supplements

Phase 1 also employs selected dietary supplements 210 that are consumed by the individual during the Phase 1 period. Today's supplement market is largely unregulated so it can be difficult to select appropriate brands. In an example embodiment, the Amare® supplements is selected for Phase 1. Amare® supplement's extremely high-quality standards and research in the nutraceutical field consistently produce high quality supplements. Their “fundamentals” pack 212, which is provided in all four phases of the recovery and resiliency program, addresses the all-important but often overlooked role of the gut-brain connection. Amare®'s studies suggest that their supplements can improve mood and reduce tension. For a good supplement regimen to work, the user must follow a regular protocol. Amare®'s labeling and instruction are extremely straightforward, helping anyone take these supplements in the right doses, at the right time, and for the right purposes.

The Amare® blends are based on ingredients that have been used for centuries to aid in a variety of ailments. The herbs and other plant extracts have been used in clinical settings in the form of nutraceuticals (natural, whole foods-based pharmaceutical alternatives). Though these supplements don't fall into that category as they do not claim to treat medical conditions, the same ingredients have been used for exactly that purpose. Amare® has published clinical research demonstrating their product's efficacy.

Rather than attempting to act on bodily systems in isolation as many pharmaceuticals do, whole food-based ingredients (like pomegranate extract and New Zealand Pine bark extract) are meant to work to enhance the body's own ability to heal itself. “Food is medicine” and by using plant extracts, Amare® harnesses food's power to heal in measured doses guided by research. Those with PTSD have hormonal imbalances and we know that one of the major remedies to normalize an individual with a hyperactive HPA (hypothalamic-pituitary-adrenal) axis is to provide the best possible nutrition to aid in their ability to regain homeostasis. Amare® supplements were the best found to aid for that specific issue. The supplement regime is straight-forward and veterans' testimonies demonstrate the supplements' value.

A specific Amare® brand supplement is Athletic Greens 512 which are supplied in Phase 4 only. While a nutrient dense diet is critical for anyone seeking better health, it's especially so with veterans seeking to manage PTSD. A primary symptom of post-traumatic stress is digestive irritation and poor nutrient absorbability: Thus a foundation of robust nutrition is central in the fight against chronic stress. While it's not possible to replace a well-rounded diet with a supplement, Athletic Greens offers the best, broad spectrum supplement to fill as many nutritional gaps as possible. During the program, participants learn the basics of good nutrition and the most offending agents in the Western diet. Each person's particular dietary needs are unique, and the “perfect” diet is impossible to find. However, built on the backbone of dietary best practices, Athletic Greens serves a robust complement to a healthy lifestyle.

Athletic Greens 512 is unique in that it offers the most complete package of supplemental nutrition that combines digestive aids, vitamin and mineral support, pre and probiotics, and adaptogenic herbs. There are hundreds of multivitamins on the market, but Athletic Greens goes a step further. As a greens powder, the supplement comes in a whole food form, meaning it's closer to the source of nutrition than a synthesized lab seen in most supplements. By using gently dried greens as the base, from chlorella to cocoa bean extract, rhodiola root to reishi mushroom powder, this blend is much more “bioavailable” than many other supplements on the market. It offers digestive enzymes to help facilitate nutrient uptake, probiotics to aid in repopulating a potentially damaged gut microbiome, and adaptogenic herbs that have long been used in Chinese medicine to aid an individual in responding to stress.

Following a year of taking Amare® brand supplements, itis important for the individual to continue their nutritional plan after the program is over. Athletic Greens is a broad-spectrum supplement that most of the population would bene fit from taking. By providing this final round of supplements, the idea is to prepare veterans finishing the recovery and resiliency program for nutritional success long after the program is over, to remind them that they can make positive change to improve their health.

Reading, Understanding and Communicating Information with Counselor, Health Coach and Members of Cohort Concerning Veteran Suicide and PTSD as a Disorder of the Veteran's Identity and Soul;

Phase 1 includes at least a first book 216. Written by the inventor, this book 216 provides the “why” behind the program. It speaks directly to those struggling with war and returning from war from the perspective of someone who's journeyed through it first-hand. At first glance, meditation, dietary supplementation, an emphasis on exercise and other aspects of R&R may seem unrelatable, unattainable, or, at worse, ineffective. The book, entitled Mission 22, paints a portrait of how progressing through the healing journey is a process that's marked by specific elements. The Mission 22 book recounts elements from real stories of combat tours and a seven-year path to healing. The recounting of the inventor's path to healing invites the recovering veteran into a real path of healing in a relatable, time relevant story. This isn't a perspective of the ancient, spear wielding, mythical soldiers of the Trojan war era: This is a story from modern combat and 21st century worldviews.

Every book 216 in the program is meant to illuminate the ways in which every aspect is related to every other. Some books place more emphasis on one element in the program, but it should be remembered that for the program to work, all elements are tied together and work synergistically. The Mission 22 book is a general survey on the healing journey in all aspects. By starting with this book, the stage is set for the veteran to commence their program of recovery and PTSD management.

In Mission 22, and in the combat veteran recovery and resiliency program generally, the inventor provides information and experiences that break down preconceptions about what the veteran, the veteran's family and society in general think about PTSD and how to heal it. It's a process that's far more than a pill taken to solve a chemical imbalance. The book also begins the process of breaking down the stigma that PTSD is a product of weakness. This, too, is a common theme in the program and accompanying books. This book describes a trailhead for a better path to healing that begins to tear down edifices of guilt and shame.

A second book 218 in Phase 1 of the combat veteran recovery and resiliency program is entitled Transitions. While the narrative doesn't speak directly to combat veterans returning from war, the national best-seller explains in plain terms that humans have an innate process we go through during times of change. In few other contexts is that as prominent as the transition between combat and home life. The book offers practical guidance in navigating life's changes that can be applied to job, relationship, location, or peacetime transitions.

This book 218 is a New York Times® bestseller. It's short, approachable, uses powerful stories, and allows people to walk through healthy change. It's used nation-wide in university and therapy settings.

Transitions is a survey on spiritual and emotional shifts during major life change. The soul aspect of the human being has a difficult time healing if shame and guilt are regular narratives that run through one's thought life. There is much to be said about positive thinking and healing. Living under the shame/guilt burden keeps the door shut on abundance and grace.

Far more than a basic “how to” or “step by step guide,” this best seller provides a map for major life changes. Suicides often occur in the in between space of war and return, or married to single, or other major shifts in what would otherwise be anchors to one's identity. Transitions puts the spotlight on the rollercoaster of emotional processing each person goes through when entering life's phase shifts. Without understanding the process of ending, transition, and new beginning, we can feel lost and overwhelmed. Veterans go from commando to salesman, Ranger to programmer. These are rough shifts that can mess with veterans' psyches. Transitions guides the veteran participants along that process with reassurance and practical application.

This program is a transition so getting veterans in the mindset that there is a method in the madness is very important up front. Other books have talked about life transitions but none of them state it as clearly as Transitions.

War and the Soul

Some embodiments of Phase 1 include, as one of the books 216, 218, War and the Soul. In the program, PTSD is seen as more than just a disorder of identity: It is a disorder of the whole person and nation. If a Soldier has PTSD, the nation has PTSD. The veterans come to understand that, through the stories in War and the Soul, they're not individually weak of character just because they've experienced trauma. It's not their weakness that causes them to experience these symptoms. Yet this is the stigma and one that often gets in the way of healing for the veteran.

The author, Edward Tick, has a wealth of experience and condensed the story of soul wounding and soul healing into fewer than 300 pages. There are bits and pieces about soul wounding in other literature, but no other book combines psychological research with frontline treatments. It was because the author studied psychology and treated Veterans that he was able to conclude that the soul is a part of the treatment. This book resonated the best. The author took very big ideas and made them workable, relatable, and tangible. He's an academic that has the ability to teach these difficult concepts well to the layperson. The self-awareness this book offers could open up a lot of doors to trust and vulnerability and acceptance so that the reader can change and heal. This book takes a close look at the aspect of the soul (emotional center) in healing.

PTSD is a commonly misunderstood condition in American culture. By and large, people think it's an individual problem someone has after being traumatized. Much of the thinking behind PTSD treatment is chemistry based. The same phenomenon drives the over-prescription of antidepressants. While chemical imbalances offer clues about how one can facilitate the healing process, author Edward Tick demonstrates that PTSD is a “soul wound.” In modern times, it can be difficult to articulate what this means as one can't explain it via the scientific method. By driving the theme that PTSD is a condition that involves not just chemistry, but one's very soul, participants can begin to understand that healing cannot simply come on the opposite side of a chemical equation.

These are very important concepts to understand at the beginning of the program. That is a large part of the reason it is included in Phase 1. It's an emotional and spiritual healing launching pad.

Engaging in Phase 1 of the Odyssey Course and in Health Coaching

A budding vocation in the field of healthcare, Health Coaching can serve to bridge the gap between a physician's prognosis and an individual's compliance. There is a significant amount of new material each participant needs to navigate through this program and a Health Coach is there to help them navigate their new understandings. The Odyssey Course is a first of its kind educational platform that teaches the participant foundational principles of health and well-being as it relates to a life with past trauma. Health Coaching pairs action with education.

Health Coaching is different from more narrowly focused coaching or life coaching. It uses a whole-body approach. While there's plenty of coaching talent to pick from, Health Coaching meshes with the crux of the program well: Whole body healing requires whole body solutions. Health Coaches appreciate the spectrum of good living from nutrition to spiritual practices. As this program encompasses holistic healing, the program needs coaches who work with these principles as well. The health coaching is done through video conferencing calls, email and phone communication, and it is paired with the online educational resource, the Odyssey Course. Health coaching plants the seeds for veterans to become more autonomous in making individual, healthy decisions.

Professional Counseling

Each individual in the program will have varying degrees of clinical needs. While R&R is not a clinically-based platform, by partnering with licensed counselors, the counselors of the program are able to both monitor and screen for individuals at a greater risk as well as offer individuals access to an extremely beneficial service or emotional processing. While other PTSD treatments largely end at counseling, within this framework, it's simply a stop along a much bigger journey.

Each participant will have a minimum of one counseling session. They'll have optional video calls over the course of the program at their discretion. Professional counseling is included in Phase 1 partially for screening purposes and partially to introduce the participant to as many relevant resources as soon as possible.

Private, Online Forum (Via Workplace) and Community Gym Memberships

Establishing community is vital for healing in many ways. This piece cannot be missed in a program aimed at healing the whole person and whole society. Workplace By Facebook® is an easily accessible platform that many users are familiar with. The gym membership must be a group-based facility like CrossFit® or mixed martial arts (“MMA”). The online forum is a place where participants can share struggles, wins, and where the program creators can take a pulse on how the cohorts are progressing. Workplace By Facebook® provides a user-friendly interface. Community connectedness and group fitness is included in each Phase of the program. Traditional gyms that don't offer the social component of CrossFit®/MMA type facilities were rejected because they lack the community aspect.

Phase 2

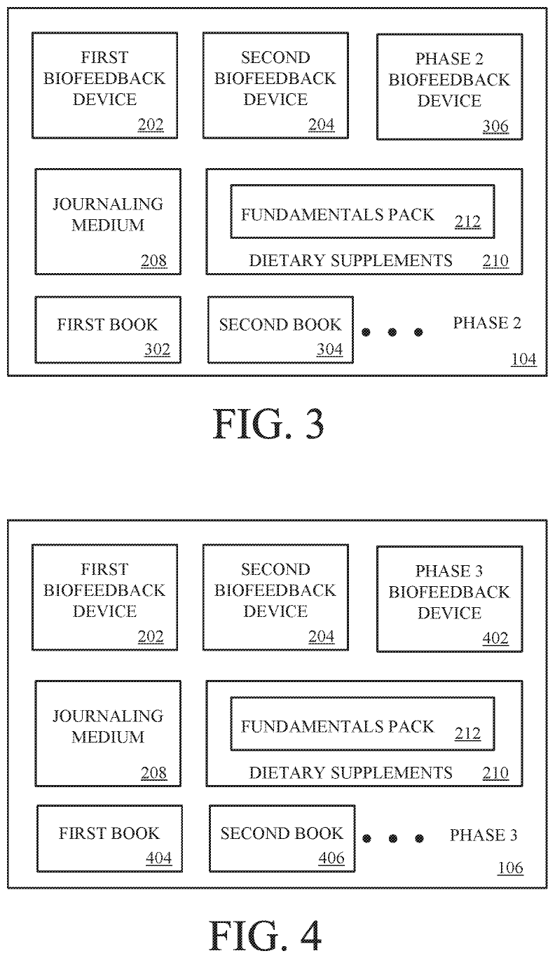

FIG. 3 is a block diagram of elements of Phase 2 of the combat veteran recovery and resiliency program 100. Phase 2 (reference numeral 104 in FIG. 1) comprises a plurality of books 302,

Phase 2 includes Warriors Return as one of the books 302. This is a sequel to War and the Soul and builds on previously studied topics. Author Edward Tick builds on War and the Soul from Phase 1 and gets more down to the business of how a Veteran can better reintegrate in American society. In War and the Soul, he gives examples of time-honored traditions such as sweat lodges and retreats back to warzones where veterans can find healing. In Warriors Return, he shifts his focus on what our culture can do to foster this sacred process.

This book takes another look at the aspect of the soul (emotional center) in healing. Concepts in War and the Soul were foundational to begin healing from combat trauma and normalizing the post-traumatic stress response. Warrior's Return builds on these topics and explores them further in the context of the warrior's return home. Other books have talked about war and emotional wounding but none of them stated it as clearly as this book does. It serves as an excellent sequel to the Phase 1 books.

The Body Keeps the Score

The Body Keeps The Score, book 304, contains information on how trauma literally reshapes both body and brain, compromising the combat veteran's capacities for pleasure, engagement, self-control and trust. By putting words to physical manifestations of PTSD, readers can peek behind the curtain of what may otherwise be considered an out of control condition. The guidance laid out in this book interleaves extremely well with the biofeedback tools the participants will be receiving at the same time. Not only will they be able to read about the physiological and psychological aspects of PTSD, they can experience first-hand how they can change for the better while measuring it with their tools. No other book complements traumatic healing with neuroscience, biology, and psychological field work. The Body Keeps The Score is often cited within the psychoanalytic community for its masterful exploration of the physical nature of trauma: It “pops the hood” on the mind-body connection and uses real world examples and neuroscience to make these connections.

As the program progresses, participants will begin to understand the nuances of PTSD symptoms, causes, and sources of healing. The book brings together years of psychological and biological research and explains with historical and contemporary examples how trauma changes the body.

Posture of Meditation

Of every calming technique available, none are matched by the practice of meditation. This practice is new for many Americans, yet ancient wisdom and modern science both validate its efficacy. This short, accessible piece is a how-to resource to help bring more life into the veteran's meditation practice. This short but powerful book explores more of the Eastern approach to this practice. This book was exactly what the program needed for that role.

After experiencing meditation first-hand and breaking down potential Western stigmas associated with the practice, we wanted to provide a more Eastern-based, easy to read guide. The halfway point in the program, namely Phase 2 vas a good milestone to introduce these concepts.

Muse®

Meditation is a spiritual practice that impacts physical and emotional health. The combat veteran recovery and resiliency program is for a particular demographic of individuals who served in the United States armed forces. Generally speaking, meditation is not a practice common among our country's service members nor among our broader culture. Phase 2 employs a biometric device 306 to facilitate the individual's meditation learning process. As science begins to validate this ancient practice, meditation devices have begun to crop up to help people get started with their own practice. Muse® is one such non-limiting example biometric device 306. It measures brainwave activity and, paired with an app and headphones, facilitates guided meditation while providing real time feedback on one's practice. For individuals new to the technique, it can be difficult to understand when one is doing it “right,” and with meditation, this can be particularly tricky to quantify. The Muse® device takes the guesswork out of the picture. Where NuCalm® is like helping the user push a boulder down-hill, Muse® sets the boulder on a level field but offers a tool for leverage.

Realtime measurable feedback is extremely important in the program's success. This biometric device 306 provides each participant proof of one's agency in their meditation practice. The biometric device 306 may optionally communicate acquired data to the analysis system 110 (FIG. 1).

Ease of use was an important feature. The inventor designed the program so that the technology used would be as seamless to the user as possible. We tested this device in house and found that it worked admirably. Muse® uses electroencephalogram technology common among neuroscientists. It translates brain activity into ambient sounds to help guide the user toward a better meditative state.

Use of Muse® follows use of NuCalm®. Muse® is a passive technology whereas NuCalm® is far more active in acting on the body's parasympathetic activity. The Muse® user has to make an effort toward stillness and the device gives gentle prompts to get them there whereas NuCalm® takes them to that place of stillness more actively. The end objective is to teach the user to be able to potentially meditate any time or place.

All the Other Elements of Phase 1 are Continued in Phases 2, 3 and 4

The Amare® supplements, the health coaching and psychological counseling, the exercise programs and the Odyssey Course are all continued throughout Phases 2, 3 and 4.

Phase 3

FIG. 4 is a block diagram of elements of Phase 3 (reference numeral 106 of the combat veteran recovery and resiliency program 100.

Heart rate variability (HRV) research has opened up a bold, new frontier in biological discovery. This technology unveils the instantaneous state of our central and autonomic nervous system. It can tell us how recovered we are from intense exercise, how synchronized our breath, mind, and hearts are, and it can also objectively measure social synchronicity.

While the Garmin® fēnix® can measure HRV to display a stress score, Phase 3 employs a different biometric device 402, such as, but not limited to, the Inner Balance® device. The Inner Balance® device 402 was specifically designed to help users enter a state of coherence. Inner Balance® is a product of research done at the HeartMath Institute. According to their scientists, “Coherent heart rhythms send signals to the brain that open up our higher brain functions. We become smarter. Practicing heart coherence for even a few minutes lifts your vibration, making it possible for your heart's intuition to inform your attitudes, discernment and choices.”

Hypervigilance keeps people stuck in “system 1 mode”, or lower brain functioning. They're in a perpetual state of reaction instead of action. By tapping into one's “heart space” or coherence, participants can create traction between “system 1 mode” and higher brain function. It gives recovering veterans access to more productive and healthful ways of thinking.

The bridge between fight-or-flight and rest-and-digest brain function often collapses when one is living with PTSD. By using the researched techniques offered by the HeartMath Institute combined with the Inner Balance® tool, this bridge can begin to repair. While it takes intentional practice by the user, Inner Balance® demonstrates that they have control of their healing. Their body will respond to healing practices chosen by the mind; Inner Balance® is the tool that illuminates this process.

The HeartMath Institute has done extensive research with individuals recovering from trauma, special operations operators, professional athletes, as well as those needing a hand in stress management. This immense body of knowledge led them to publish several books, two of which are included in the program, and this device by itself has huge potential in realigning the user with their regenerative emotions.

By clipping on a veteran's ear lobe and sending HRV data to the app, this device measures coherence and provides real time feedback. It produces a graph that is easy to read and understand. One can see how meditative practice has an immediate impact on heart rhythms. The educational component of Inner Balance®—namely the two companion books (as will be discussed below) as well as online resources—gives the Veteran that much more confidence and buy-in.

Here is yet another example of synergy at work—Inner Balance® working with the meditation tools provided in Phases 1 and 2 to create a greater result than the sum of its parts.

Meditation is a spiritual practice that impacts physical and emotional health. Learning the essence of meditation through the Posture of Meditation book in the preceding phase as well as the other two meditation devices serve as excellent prerequisites for Inner Balance®. The numbers and methods of Inner Balance® will make much more sense after nearly six months of meditative practice and experience.

The accuracy and research that are wrapped up in Inner Balance® and its companion app are unparalleled in the industry.

Achilles in Vietnam

The theme of the warrior's good return home marks the essence of the recovery and resiliency program. This book 404 continues the conversation through metaphor and another perspective that tells a story similar to the books of Edward Tick.

This tragic survey of the American Warrior's journey through one of the nation's darkest conflicts brings to light what happens when the Warrior is not honored in his pursuits. The author expertly layers the timeless epic of the Iliad with the devastation of the Vietnam war. Men have been fighting wars and honoring a sacred process therein for centuries. It is really only with the advent of 20th century American fighting methods that we've totally lost touch with respecting our fighters as those in Homer's story did. The result left thousands of Vietnam Veterans in the margins of society, without a real place to call home. Their loss of purpose led to a death by suicide count that would nearly triple the size of the Vietnam Memorial wall.

This theme and storytelling style are relatable to modern warriors as it tells an American story paired with an ancient Greek one. Here, we explore more of a soulful/spiritual/emotional journey of warriors who either journey poorly or journey well through the throes of war.

Atomic Habits

Along with instruction on how to begin a meditation practice, incorporate healthier foods, and a better exercise regime, Atomic Habits 406 helps its reader understand how to form these into habits that will help each veteran get the most out of the program long after it has come to an end.

Many effects of PTSD lead to poor habit formation. For this program to work, individuals must ingrain healthier habits that compound to create an abundant life. Avoidance behavior, substance abuse, and overreactions to benign stimulations are but some difficulties these Veterans and their families deal with. Atomic Habits provide straightforward guidance on behavior change. It offers an extremely accessible “toolkit” paired with entertaining examples and vignettes about what each tool looks like practically.

Habit change can impact every aspect of well-being. While this book might have been placed in any phase, it appears here because, before habit change begins, participants must have a solid “why” to work from. If an individual is struggling with doubt and grief and everything else that comes with PTSD, reading about new habits right out of the gate is likely going to be less effective.

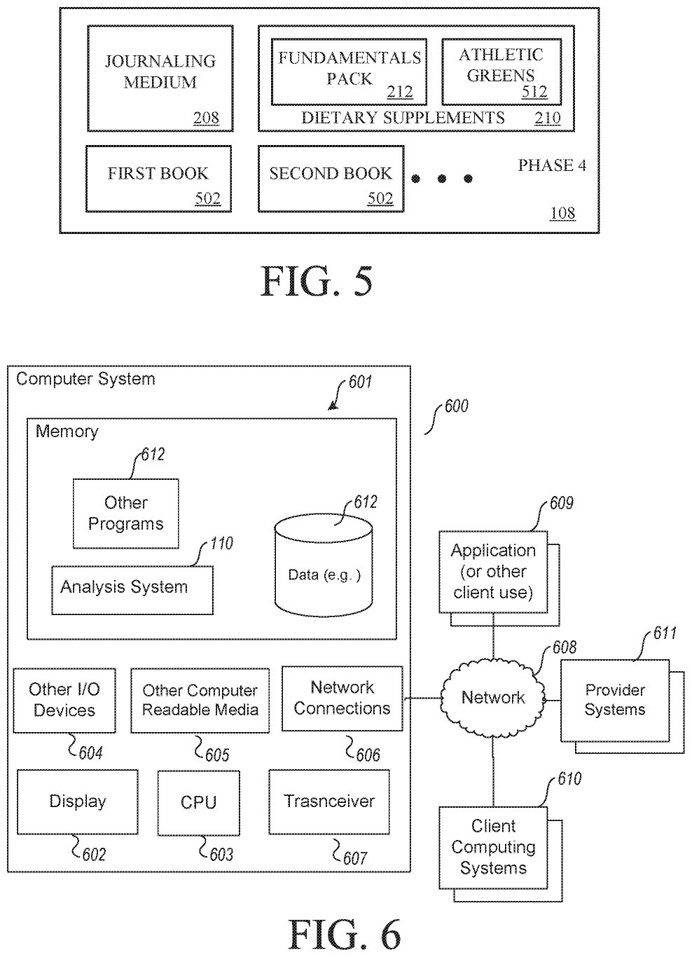

FIG. 5 is a block diagram of elements of Phase 4 of the combat veteran recovery and resiliency program 100. Phase 4 includes the dietary supplements 210, the journaling medium 208, and a plurality of books 502.

Transforming Anger and Transforming Stress (Phases 3 and 4, Respectively):

These two books 502 are written with the Inner Balance® user in mind. Sharing the HeartMath Institute's recent discoveries in neuroscience and the impact of heart function on emotional health, these books provide practical guides in managing one's emotions. The techniques and science are based on the understanding of the heart as a real, emotional carrying organ. If the Inner Balance® device is the speedometer, these books are the car's user manual These give the “why” behind the powerful tool that is found in the Inner Balance® devce. No other books exist that are so specific to those needs, Giving more tools and science behind meditative practices shores up a well-rounded routine to cairn and center.

These are somewhat advanced level topics and so are included in Phases 3 and 4. The user should understand the basics of meditation before getting in deep with the topics addressed in these two books.

Odysseus in America

The theme of the warrior's good return home marks the essence of recovery and resiliency program. This book 502 continues the conversation through metaphor and another perspective that tells a similar story as Edward Ticks' books.

This book 502 is Achilles in Vietnam's Act 2 and explores the Veteran's journey home. In Homer's epic, the war and the trip back home took 10 years each, illustrating how the Warrior is a changed human being upon arrival back at the home front. Again, our American culture has forgotten this important transition and replaced it with an annual parade and hardware store discounts. The story follows similar design patterns as Act 1 and helps explain that shifting back into home life is not a process to be taken lightly. Our ancestors knew this and this book serves as a reminder to the modern, Western age.

In this book 502, the author and reader explore more of a soulful/spiritual/emotional journey of warriors who either journey poorly or journey well through the return home from war. This is a companion piece meant to be read after Achilles in Vietnam, which appears in Phase 3.

Power of Positive Thinking

This best seller 502 addresses one of the most heinous sources of despair and suicide: our own negative inner narrative. Dr Peale teaches his readers how to master their thought life, overcome self-doubt, and visualize solutions in the midst of problems. This book is particularly appropriate for our program participants. So much of our fear, worry, and health declines are rooted in our thinking. By removing the stigma and outlining healthy methods of thinking, intrepid readers can experience life freed from the shackles of stinking thinking that often accompany PTSD and TBI. This is a time-honored best seller that uniquely fuses the concepts of practicing good thinking and biological responses.

The nature of beliefs and physical manifestations is a tricky but important topic. This concept is teased throughout the program but is put into more specific words in this book. Where the previous phases cultivate this understanding, this book begins to plant and grow the seeds.

Which Way is Your Claymore Facing: An Operational Manual

During his own time with the Rhodesian military, author Barry Zworestine saw and felt firsthand how difficult it can be to leave the soldier's mind-set behind. In this book, the author explains how to begin with small changes and embrace the uncertainty of your new life, to tolerate different kinds of emotional discomfort. He encourages the veteran to keep moving forward, to stop letting impulses get the better of them, to refrain from blaming themselves or others and to take the time to enjoy the good moments when they come. In short, it is a “how to resume normal life again” manual 502.

FIG. 6 is a block diagram of the analysis system 110 of FIG. 1. in an operating environment 600. The analysis system 110 may be used to practice embodiments of a combat recovery and resiliency program described herein. Note that one or more general purpose virtual or physical computing systems suitably instructed or a special purpose computing system may be used to implement an analysis system 110. Further, the analysis system 110 may be implemented in software, hardware, firmware, or in some combination to achieve the capabilities described herein.

Note that one or more general purpose or special purpose computing systems/devices may be used to implement the described techniques. However, just because it is possible to implement the analysis system 110 on a general purpose computing system does not mean that the techniques themselves or the operations required to implement the techniques are conventional or well known.

The computing system 600 may comprise one or more server and/or client computing systems and may span distributed locations. In addition, each block shown may represent one or more such blocks as appropriate to a specific embodiment or may be combined with other blocks. Moreover, the various blocks of the analysis system 110 may physically reside on one or more machines, which use standard (e.g., TCP/IP) or proprietary inter-process communication mechanisms to communicate with each other.

In the embodiment shown, computer system 600 comprises a computer memory (“memory”) 601, a display 602, one or more Central Processing Units (“CPU”) 603, Input/Output devices 604 (e.g., keyboard, mouse, CRT or LCD display, etc.), other computer-readable media 605, one or more network connections 606, and an optional transceiver 607 configured to communicate using wireless signals with the various biometric devices disclosed herein. The analysis system 110 is shown residing in memory 601. In other embodiments, some portion of the contents, some of, or all of the components of the analysis system 110 may be stored on and/or transmitted over the other computer-readable media 605. The components of the analysis system 110 preferably execute on one or more CPUs 603 and manage the use of the analysis system 110, as described herein. Other code or programs 630 and potentially other data repositories, such as data repository 606, also reside in the memory 601, and preferably execute on one or more CPUs 603. Of note, one or more of the components in FIG. 6 may not be present in any specific implementation. For example, some embodiments embedded in other software may not provide means for user input or display.

The computing system 600 may comprise one or more server and/or client computing systems and may span distributed locations. In addition, each block shown may represent one or more such blocks as appropriate to a specific embodiment or may be combined with other blocks. Moreover, the various blocks of the analysis system 110 may physically reside on one or more machines, which use standard (e.g., TCP/IP) or proprietary inter-process communication mechanisms to communicate with each other.

In a typical embodiment, the analysis system 110. Other and/or different modules may be implemented in alternative embodiments. In addition, the analysis system 110 may interact via a network 608 with application or client code 609 that uses results computed by the analysis system 110, may interact with one or more client computing systems 610, and/or one or more third-party information provide systems 611, such as various purveyors and/or users of information used in a data repository 612 and/or generated by the analysis system 110. Also, of note, the data repository 612 may be provided external to the computing system 600 as well, for example in a world wide web knowledge base or other memory system that is accessible over one or more networks 608.

In an example embodiment, components/modules of the analysis system 110 are implemented using standard programming techniques. However, the analysis system 110 may be implemented in other forms, such as object-oriented, distributed, etc. implementations. For example, the analysis system 110 may be implemented as a “native” executable running on the CPU 603, along with one or more static or dynamic libraries. In other embodiments, the analysis system 110 may be implemented as instructions processed by a virtual machine. A range of programming languages known in the art may be employed for implementing such example embodiments, including representative implementations of various programming language paradigms, including but not limited to, object-oriented (e.g., Java, C++, C#, Visual Basic.NET, Smalltalk, and the like), functional (e.g., ML, Lisp, Scheme, and the like), procedural (e.g., C, Pascal, Ada, Modula, and the like), scripting (e.g., Perl, Ruby, Python, JavaScript, VBScript, and the like), and declarative (e.g., SQL, Prolog, and the like).

The embodiments described above may also use well-known or proprietary, synchronous or asynchronous client-server computing techniques. Also, the various components may be implemented using more monolithic programming techniques, for example, as an executable running on a single CPU computer system, or alternatively decomposed using a variety of structuring techniques known in the art, including but not limited to, multiprogramming, multithreading, client-server, or peer-to-peer, running on one or more computer systems each having one or more CPUs. Some embodiments may execute concurrently and asynchronously and communicate using message passing techniques. Equivalent synchronous embodiments are also supported. Also, other functions could be implemented and/or performed by each component/module, and in different orders, and in different components/modules, yet still achieve the described functions.

In addition, programming interfaces to the data stored as part of the analysis system 110 (e.g., in the data repositories 612) can be available by standard mechanisms such as through C, C++, C#, and Java APIs; libraries for accessing files, databases, or other data repositories; through scripting languages such as XML; or through Web servers, FTP servers, or other types of servers providing access to stored data. The analysis system 110 may be implemented as one or more database systems, file systems, or any other technique for storing such information, or any combination of the above, including implementations using distributed computing techniques.

Alternatively, the analysis system 110 may be implemented in a distributed environment comprising multiple, even heterogeneous, computer systems and networks. Different configurations and locations of programs and data are contemplated for use with techniques of described herein. In addition, the [server and/or client] may be physical or virtual computing systems and may reside on the same physical system. Also, one or more of the modules may themselves be distributed, pooled or otherwise grouped, such as for load balancing, reliability or security reasons. A variety of distributed computing techniques are appropriate for implementing the components of the illustrated embodiments in a distributed manner including but not limited to TCP/IP sockets, RPC, RMI, HI IP, Web Services (XML-RPC, JAX-RPC, SOAP, etc.) and the like. Other variations are possible. Also, other functionality could be provided by each component/module, or existing functionality could be distributed amongst the components/modules in different ways, yet still achieve the functions of an analysis system 110.

Also the example analysis system 110 may be implemented in a distributed environment comprising multiple, even heterogeneous, computer systems and networks. Different configurations and locations of programs and data are contemplated for use with techniques of described herein. In addition, the [server and/or client] may be physical or virtual computing systems and may reside on the same physical system. Also, one or more of the modules may themselves be distributed, pooled or otherwise grouped, such as for load balancing, reliability or security reasons. A variety of distributed computing techniques are appropriate for implementing the components of the illustrated embodiments in a distributed manner including but not limited to TCP/IP sockets, RPC, RMI, HI IP, Web Services (XML-RPC, JAX-RPC, SOAP, etc.) and the like. Other variations are possible. Also, other functionality could be provided by each component/module, or existing functionality could be distributed amongst the components/modules in different ways, yet still achieve the functions of the analysis system 110.

Furthermore, in some embodiments, some or all of the components of the analysis system 110 may be implemented or provided in other manners, such as at least partially in firmware and/or hardware, including, but not limited to one or more application-specific integrated circuits (ASICs), standard integrated circuits, controllers executing appropriate instructions, and including microcontrollers and/or embedded controllers, field-programmable gate arrays (FPGAs), complex programmable logic devices (CPLDs), and the like. Alternatively, or additionally, the analysis system 110 may be implemented with, and/or as part of, other programs 612. Some or all of the system components and/or data structures may also be stored as contents (e.g., as executable or other machine-readable software instructions or structured data) on a computer-readable medium (e.g., a hard disk; memory; network; other computer-readable medium; or other portable media article to be read by an appropriate drive or via an appropriate connection, such as a DVD or flash memory device) to enable the computer-readable medium to execute or otherwise use or provide the contents to perform at least some of the described techniques. Some or all of the components and/or data structures may be stored on tangible, non-transitory storage mediums. Some or all of the system components and data structures may also be stored as data signals (e.g., by being encoded as part of a carrier wave or included as part of an analog or digital propagated signal) on a variety of computer-readable transmission mediums, which are then transmitted, including across wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of this disclosure may be practiced with other computer system configurations.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A combat veteran recovery and resiliency program method that heals the emotional scars of the soul of an individual suffering from post traumatic stress disorder (PTSD) or combat related traumatic brain injury (TBI), wherein the combat veteran recovery and resiliency program is defined by four sequential phases, wherein each of the four phases comprises:
an emotional healing aspect,
a physical healing aspect,
a spiritual healing aspect, and
a nutritional healing aspect,
wherein each of the four sequential phases occur in a logical progression,
wherein each of the four sequential phases integrate with the emotional healing aspect, the physical healing aspect, the spiritual healing aspect, and the nutritional healing aspect for each phase, and
wherein the combat veteran recovery and resiliency program results in emotional healing of the individual that is greater than the sum of the individual aspects.

2. The method of claim 1 wherein the emotional healing aspects of the program comprises:
engaging in at least one session of psychological counseling;
taking medication as prescribed by a physician or psychiatrist;
the combat veteran relating and sharing the combat veteran's experiences within a cohort;
using of a biofeedback device that induces brain wave changes to give the veteran a brain wave pattern rich in alpha and theta waves;
using a biofeedback device that monitors the combat veteran's reactions to stress and monitors sleep quality and length of sleep;
using a biofeedback device that measures brain activity and provides real-time feedback on brain state;
use of a biofeedback device that measures heart rhythm patterns;
reading information written by combat veterans about their recovery from PTSD and TB;
reading information about transitions and changes in life;
reading information about positive thinking; and
journaling by the combat veteran to capture the veteran's own emotional reactions, daily routines and changes in emotional and spiritual wellbeing.

3. The method of claim 1 wherein the physical healing aspect comprises:
a gym membership;
fitness coaching;
participation as a member of a cohort in a fitness program; and
use of at least one biofeedback device that provides measurable metrics that track the combat veteran's physical healing progress.

4. The method of claim 1, wherein the spiritual healing aspect includes at least one activity chosen to work concomitantly with the aspects of the program, wherein the use of at least one of:
using a biofeedback device,
journaling, spiritual changes, and
learning how to meditate.

5. The method of claim 1, wherein the nutritional healing aspect includes managing eating by the individual:
eating dietary supplements; and
eating athletic greens.

6. The method of claim 1, wherein a first phase of the four sequential phases includes the following aspects:
using a first biofeedback device to stimulate and cause parasympathetic nervous system activation to induce a meditative alpha and theta brain wave rich state;
using a second biofeedback device to track recovery from stress and to perform sleep analysis, and sending the data generated to the health coach for review and analysis;
engaging in physical exercise program with members of cohort at a gym or fitness center;
recording spiritual and emotional growth in a journal designed to capture these experiences;
including dietary supplements provided;
reading, understanding and communicating information with health coach and members of cohort members concerning veteran suicide and PTSD as a disorder of the veteran's identity and soul and concerning modern society's misconception about PTSD, and material setting forth steps that humans must take when transitioning from one life situation to another;
building a support system through inclusion of some or all the cohort's members and creating a support system chart establishing a plan for the next time the combat veteran needs help; and
engaging in Phase 1 of the Odyssey Course.

7. The method of claim 1, wherein a second phase of the four sequential phases includes:
reading about transforming the wounds of war into sources of wisdom, honor and growth;
reading information on how trauma literally reshapes both body and brain, compromising the individual's capacities for pleasure, engagement, self-control and trust;
learning to meditate using a third biofeedback device that measures brain activity and gives real-time feedback on brain wave state to help guide the meditation;
receiving guidance about the importance of body position during meditation;
including dietary supplements provided in diet;
engaging in physical exercise program with members of a cohort at a gym or fitness center; and
engaging in Phase 2 of the Odyssey Course.

8. The method of claim 1, wherein a third phase of the four sequential phases includes:
using a heart rate variability biofeedback device to gauge how the individual's heart responds to and recovers from stress;
reading information on transforming anger and using this information in conjunction with the heart rate variability monitor to let go of rage, frustration and irritation;
reading information on how combat veterans were regarded in the time of the Iliad and contrasting that with how combat veterans are regarded in modern day society;
reading information and practicing using the tools disclosed therein on changing habits to identify areas of growth and new understanding; and
including dietary supplements provided in diet;
engaging in physical exercise program with members of a cohort at a gym or fitness center; and
engaging in Phase 3 of the Odyssey Course.

9. The method of claim 1, wherein a fourth phase of the four sequential phases includes the combat veteran:
reading information on eliminating self-doubt, worry, stress and resentment and how to climb above problems to visualize solutions and then attain them;
reading information on how to leave the soldier's mind-set behind by beginning with small changes, embracing the uncertainty of life, tolerating emotional discomfort, not acting on impulses and refrain from blaming self or others;
using the story of the Odyssey to illuminate the pitfalls that trap many veterans on the road back to civilian life;
practicing meditation without the use of any feedback devices;
supplementing diet with athletic greens supplements; and
engaging in physical exercise program with members of cohort at a gym or fitness center.
